(19) United States
(12) Patent Application Publication
(10) Pub. No.: US 2010/0256689 A1
(43) Pub. Date: Oct. 7, 2010

(54) SLIDER LOCKING MECHANISM FOR PEDICLE SCREWS

(75) Inventor: Keith E. Miller, Germantown, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/416,953

(22) Filed: Apr. 2, 2009

(51) Int. Cl. A61B 17/86

(52) U.S. Cl. 606/305; 606/301

(57) ABSTRACT

Systems, devices and methods are provided for locking a spinal rod in a top loading pedicle screw. In one form, the device includes a slider geometrically shaped to secure the spinal rod in the bone screw in its final resting position. Engagement pins located on the slider mate with notches located on the bone screw that both provisionally, and eventually, lock the spinal rod in the bone construct. The device replaces threaded set screw designs that have been known to incur cross threading problems. Thus, the device can also make spinal rod tightening faster and easier when compared to a threaded set screw.

### BACKGROUND OF THE INVENTION

It has become accepted for certain spinal deformities to stabilize the relative position of vertebrae for at least some limited period of time. Anchor members are attached onto vertebral bodies, such as in the case of laminar hooks, or are implanted into the bodies using screw members. The anchors generally include devices to hold a rod such as a hook or pedicle screw that forms an integral part of a bone anchor. Adjacent bone anchors customarily hold respective vertebrae relative to a stabilizer, i.e. a plate or a rod.

In describing the corrective actions for a spinal deformity using bone anchors, it is helpful to keep the biological climate in mind. It is useful not only to achieve desired load limitations and stabilization characteristics, but also to design a series of components and respective instrumentation that is as easily manipulated and as quickly assembled as possible, which is as non-obtrusive into the biological climate as possible, and which is designed with the goal of avoiding failure. A surgeon may have limited physical access to the surgical site as well as obscured surgical visibility. It is helpful to avoid the pieces or filings which could fall into a wound site. It is also important to design a system that can be manipulated by a gloved surgeon that allows flexibility in surgical technique, and options for corrective instrumentation.

The current invention provides a top loading pedicle screw having a slider that slides into position to capture a spinal rod within a rod channel portion of the top loading pedicle screw.

The invention also provides a locking mechanism that replaces current threaded set screws used on many types of pedicle screws. This invention also eliminates cross threading.

The present invention relates generally to treatment of the spinal column, and more particularly, relates to a locking mechanism for top loading pedicle screws. Top loading pedicle screws have an opening in the top of the screw in which a spinal rod is placed. That opening is typically closed with a set screw that in turn tightens onto the spinal rod. Cross threading of the set screw is a potential issue with these types of designs. The current design includes a slider that is pushed into place instead of being threaded into place like a set screw. This slider avoids the cross threading issue.

### SUMMARY OF THE INVENTION

While the actual nature of the invention covered herein can only be determined with reference to the claims appended hereto, certain forms of the invention that are characteristic of the preferred embodiments disclosed herein are described briefly as follows.

In one form of the present invention, a top loading pedicle screw is provided. The top loading pedicle screw includes a slot formed into its rod channel portion to receive a locking mechanism. The locking mechanism is a slider that is designed to slide into a slot from the top, and, with an initial translation, provisionally locks a spinal rod into the rod channel portion. Further urging of the slider into the slot firmly secures and locks the spinal rod in place.

Further embodiments, forms, features, aspects, benefits, objects and advantages of the present invention will become apparent from the detailed description and figures provided herewith.

### BRIEF DESCRIPTION OF THE FIGURES

FIG. 1 is a perspective view of an embodiment of the slider of the present invention.

FIG. 2 is a cross-sectional view of the invention.

FIG. 3 is a cross-sectional view of the embodiment depicted in FIG. 2 after provisional capture of a spinal rod.

FIG. 4 is a cross-sectional view of the embodiment depicted in FIG. 2 after locking the spinal rod in the pedicle screw.

FIG. 5 is a cross-sectional view of another embodiment of the invention including a single elongated notch.

FIG. 6A is a cross-sectional view of another embodiment of the invention.

FIG. 6B is an embodiment of a slider for the invention described in FIG. 6A.

FIG. 7A is a cross-sectional view of another embodiment of the invention.

FIG. 7B is a detail view of the slot described in FIG. 7A.

FIG. 8 is another embodiment of the invention that uses a single elongated slot with first and second pairs of engagement pins.

FIG. 9 is another embodiment of the invention illustrated in FIG. 8 with the notch including a ramped downward portion.

### DESCRIPTION OF THE ILLUSTRATED EMBODIMENTS

For the purposes of promoting an understanding of the principles of the invention, reference will now be made to the embodiments illustrated in the drawings, and specific language will be used to describe the same. It will nevertheless be understood that no limitation on the scope of the invention is intended. Any alterations and further modifications in illustrated devices and described methods and further applications of the principle of the invention as disclosed herein are contemplated, as would normally occur to one skilled in the art to which the invention relates.

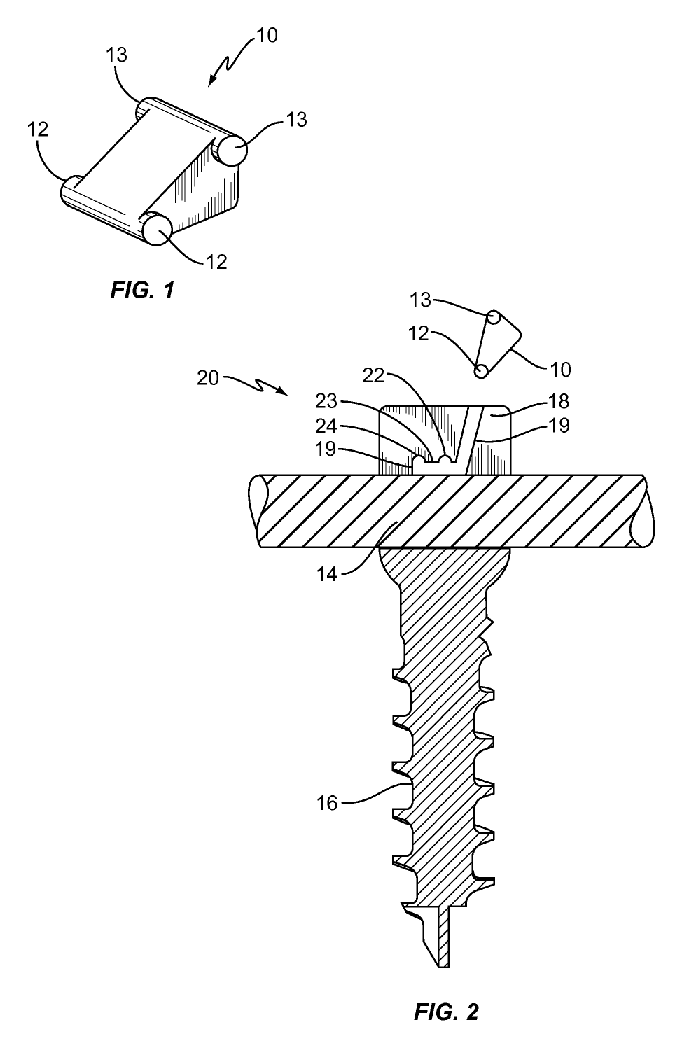

In FIG. 1, a perspective view of an embodiment of the invention is illustrated. A slider 10 is illustrated with a geometrical wedge shape. It is noteworthy that the geometrical shape of the slider 10 can be of any dimensions that will enable a securing of a spinal rod 14 into a top loading pedicle screw 16 as described in this invention (see FIG. 2). The slider 10 includes a first pair of engagement pins 12 and a second pair of engagement pins 13. The two pairs of engagement pins 12,13 are used to facilitate urging of the slider 10 into the bone screw 16 from the top portion 23 of the top loading pedicle screw 16.

FIG. 2, illustrates an initial positioning of a locking mechanism 20 wherein the slider 10 is positioned for insertion into the top loading pedicle screw 16 at a top portion 23. A slot 19 is positioned on an inside surface 18 of a rod channel portion 17 to facilitate travel of the engagement pins 12,13 during a locking procedure. A first pair of notches 22 and a second pair of notches 24 are located at the distal end 25 of the slot 19. The first pair of notches 22, and the second pair of notches 24 are used to secure the engagement pins 12,13 in the slot 19 during a preliminary securing of the spinal rod 14 into the top loading pedicle screw 16. This preliminary securing of the spinal rod 14 is a provisional locking of the spinal rod 14 in the top loading pedicle screw 16. It is to be understood that the slot 19 is located on both inside surfaces 18 of the rod channel portion 17.

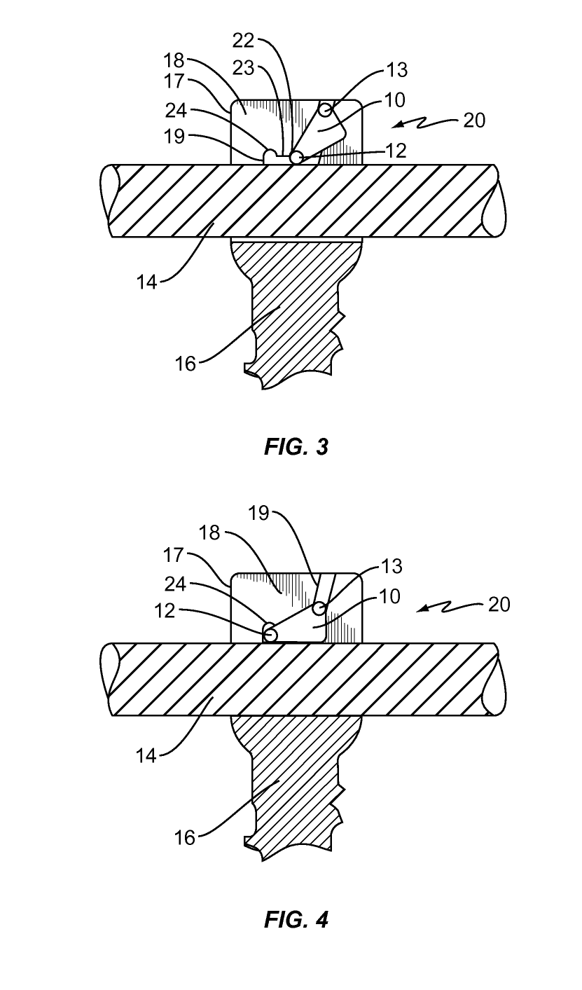

FIG. 3 illustrates a cross-sectional view of the embodiment depicted in FIG. 2 wherein the slider 10 has been urged into an initial position in the slot 19. There is a gap 33 between the spinal rod 14 and the rod channel portion 17. This gap diminishes as the spinal rod 14 is locked into position within the top loading pedicle screw 16. The first pair of engagement pins 12 can be seen interacting with the first pair of notches 22. The slider 10 includes a second pair of engagement pins 13 located at the beginning of the slot 19. In this position, the slider 10 has provisionally locked the spinal rod 14 into the rod channel portion 17. Minimal translation of the spinal rod 14 is still possible, but substantial sliding is restricted.

In FIG. 4, the slider 10 is in a fully locked position. Further urging of the slider 10 into the slot 19 has moved the first pair of engagement pins 12 beyond notches 22 (not shown) to mate with the second pair of notches 24 and the second pair of engagement pins 13 are now located further down the slot 19. Alternatively, the second pair of engagement pins 13 can engage in the first pair of notches 22 (not shown) and the first pair of engagement pins 12 can seat in the second pair of notches 24. In either of these positions, a wedge-shape design of the slider 10 along with the upward force of the spinal rod 14 has fully engaged and locked the spinal rod 14 into the rod channel portion 17 of the top loading pedicle screw 16. No further manipulation of the spinal rod 14 within the rod channel portion 17 of the top loading pedicle screw 16 is possible.

Thus, by sliding and urging the slider 10 down the slot 19 located in the rod channel portion 17 of the top loading pedicle screw 16 a spinal rod 14 can be provisionally locked and eventually, secured. The slider 10 in this embodiment with the wedge shape design facilitates the locking described above. However, any geometrical shape of the slider 10 can be used, so long as it provisionally, and finally fully locks and secures the spinal rod 14 in the rod channel portion 17.

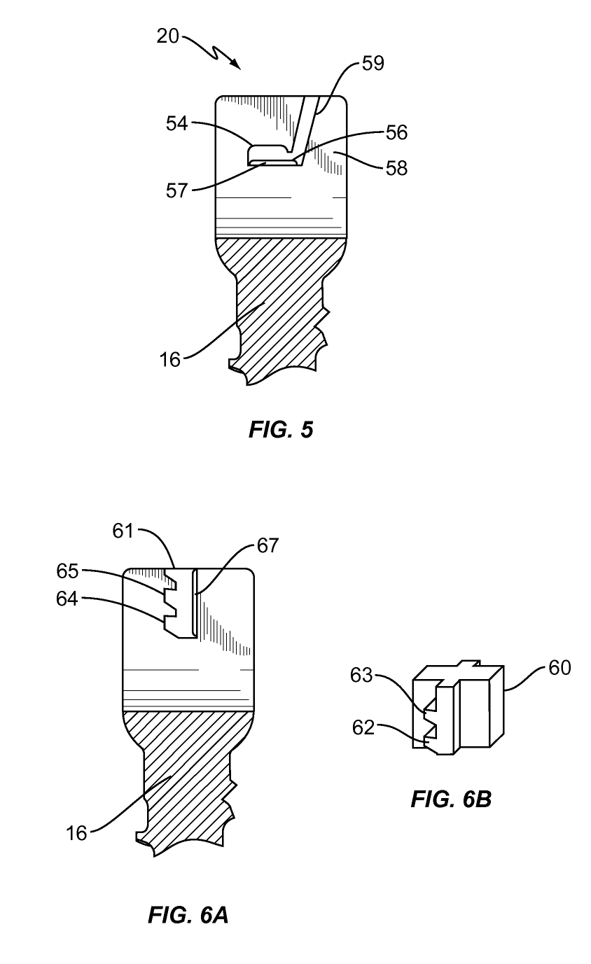

FIG. 5 illustrates a cross-sectional view of another embodiment of the invention. In this figure, a single notch 54 is connected to the slot 59. This notch 54 can be used to fit both sets of engagement pins (not shown). A front ledge 55 of the notch 54 holds the two positions of the slider (not shown). A protrusion 56 may extend along the bottom of the slot 59 below the notch 54 to create a snap fit. The protrusion 56 may be of an elastomeric material 57. The protrusion 56 may also be a spring mechanism (not shown) that creates a snap fit when the engagement pins (not shown) are slid down the slot 59 into the notch 54 from an provisional locking position of the spinal rod (not shown) to a final locking position.

In FIGS. 6A and 6B a ratcheting slot embodiment of the invention is illustrated. FIG. 6B illustrates a slider 60 that includes two pairs of engagement pins 62, 63 of a ratcheting design. The two pairs of engagement pins 62, 63 are slid down a slot 61 that includes a matching set of first and second pairs of notches 64,65(see FIG. 6A). A deformable elastomeric material 67, may be positioned in the slot 61 to help promote a snap fit as the slider 60 is slid down the slot 61. The deformable elastomeric material 67, could also be a spring mechanism (not shown). Alternatively, the deformable elastomeric material 67 may be located on a slider surface 66. The notches 64, 65 are designed to include a ratchet design (see FIG. 6A). Thus, during the provisional locking the spinal rod (not shown) is pushing the slider 60 upward. The first pair of notches 65 provides a provisional locking of the spinal rod (not shown) and the second pair of notches 64 provides a full and complete locking of the spinal rod (not shown). Thus, the ratchet design of the notches 64, 65 keeps the slider 60 in place on the top loading pedicle screw 16.

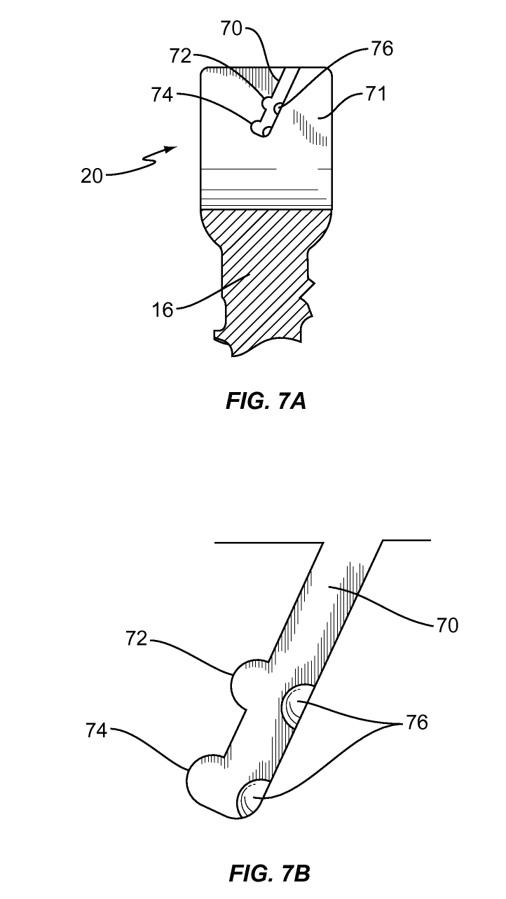

FIGS. 7A and 7B describe yet another embodiment of the invention. In this cross-sectional drawing the slot 70 is located on the inside surface 73 of the rod channel portion 71 of the top loading pedicle screw 16 at a less than vertical but greater than horizontal angle (see FIG. 7A). The slot 70 includes first and second pairs of notches 72,74 for provisional capture and locking of the spinal rod (not shown), respectively. At least one protrusion 76 (see FIG. 7B) may be available in the slot 70 to create a snap fit as the slider (not shown) is slid down the slot 70 and engages the notches 72,74. In the illustrated figure, a pair of protrusions 76 can be seen. The protrusions 76 may also be a spring mechanism (not shown). Elastomeric materials may also be used for the protrusions 76.

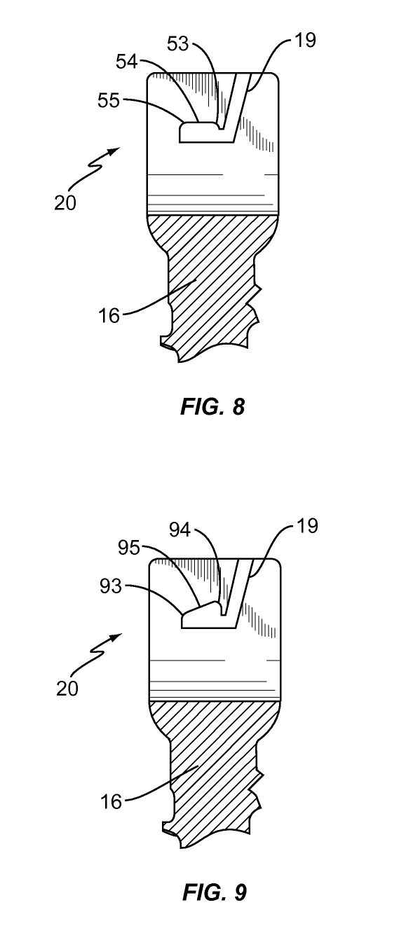

FIG. 8 describes an embodiment of the invention that uses a single notch 54 for provisional locking of the spinal rod (not shown). The first set of engagement pins (not shown) are slid down the slot 19 and engage a front portion 53 of the notch 54 for provisional locking. As the slider (not shown) is slid further down the slot 19 the second set of engagement pins (not shown) slide into the front portion 53 of the notch 54 and thus lock the spinal rod (not shown) into position. The first set of engagement pins (not shown) extend further to a distal end 55 of the notch 54 and may even contact the distal end 55.

In FIG. 9, an embodiment of the invention is illustrated that includes a slot 19 with a notch 94 that is tapered. As the first and second pairs of engagement pins (not shown) are slid down the slot 19, initial rod capture occurs at the notch 94. As the engagement pins (not shown) are slid further past the notch 94 the first pair of engagement pins (not shown) slide along the tapered portion 95 and stop at a distal end 93 of the notch 94 thus locking the spinal rod (not shown) into position. The second pair of engagement pins (not shown) may stop at the notch 94 or along the tapered portion 95.

Any theory, mechanism of operation, proof, or finding stated herein is meant to further enhance understanding of the present invention, and is not intended to make the present invention in any way dependent upon such theory, mechanism of operation, proof or finding. It should be understood that while the use of the word preferable, preferably or preferred in the description above indicates that the feature so described may be more desirable, it nonetheless may not be necessary, and embodiments lacking the same may be contemplated as within the scope of the application, that scope being defined by the claims that follow. In reading the claims, it is intended that when words such as “a”, “an”, “at least one”, and “at least a portion” are used, there is no intention to limit the claim to only one item unless specifically stated to the contrary in the claim. Further, when the language “at least a portion” and/or “a portion” is used, the item may include a portion and/or the entire item unless specifically stated to the contrary.

While the application has been illustrated and described in detail in the drawings and foregoing description, the same is to be considered as illustrative and not restrictive in character, it being understood that only the select embodiments have been shown and described and that all changes, modifications and equivalents that come within the spirit of the invention as defined herein or by any of the following claims are desired to be protected.

What is claimed is:

1. A locking mechanism for a top loading pedicle screw, comprising:
a top loading pedicle screw adapted to receive a slider; and
a slider that engagingly attaches to the top loading pedicle screw.

2. The locking mechanism of claim 1, wherein a rod channel portion is formed in the top loading pedicle screw wherein the rod channel portion is adapted to receive the slider.

3. The locking mechanism of claim 2, further comprising engagement pins located on the slider, wherein the rod channel portion includes an inside surface, the inside surface forms a slot configured to receive the engagement pins.

4. The locking mechanism of claim 3, wherein the engagement pins on the slider are configured as a first pair of engagement pins and a second pair of engagement pins, the slot includes a first notch and a second notch configured to receive the first and second pairs of engagement pins.

5. The locking mechanism of claim 3, wherein the slider and the slot are configured such that the first pair of engagement pins consecutively engages with the first and second notches.

6. The locking mechanism of claim 5, wherein the slot has a distal end and a proximal end, the first pair of engagement pins is positioned at the distal end of the slot, and the second pair of engagement pins is positioned at the proximal end of the slot when sequentially the slider is inserted in a final locked position in the top loading pedicle screw.

7. A system for locking a spinal rod in a top loading pedicle screw, comprising:
a pedicle screw with a rod channel portion, and wherein a slot is positioned on an inner surface on both sides of the rod channel portion; and
a slider that slides into the slot in the rod channel portion.

8. The system of claim 7, wherein the slider includes one pair of engagement pins that slide along the slot.

9. The system of claim 8, wherein the slot has a bottom and the slot includes a protrusion at the bottom.

10. The system of claim 9, wherein the protrusion includes a spring mechanism.

11. The system of claim 9, wherein the protrusion comprises a deformable elastomeric material and the protrusion extends along the entire bottom of the slot.

12. The system of claim 7, wherein the slot includes one notch and the slider includes an engagement pin and wherein the notch is for capture of the engagement pin.

13. The system of claim 12, wherein the slot is configured to ramp downward from a proximal end of the notch to a distal end of the notch.

14. The system of claim 13, wherein the slot includes at least one protrusion to facilitate a snap-fit when an engagement pin interacts with the proximal and distal ends of the notch.

15. The system of claim 14, wherein the protrusion includes a spring mechanism.

16. The system of claim 14, wherein the protrusion includes a deformable elastomeric material.

17. The locking mechanism of claim 4, wherein the slot includes a ratchet design.

18. The locking mechanism of claim 17, wherein the first and second pairs of engagement pins are a ratchet design and engage the notches in the slot.

19. The locking mechanism of claim 17, wherein the slot includes a deformable elastomeric material positioned opposite the notches.

20. The locking mechanism of claim 17, wherein the slider has a surface and the surface includes a deformable elastomeric material.

21. The locking mechanism of claim 19, wherein the deformable elastomeric material includes a spring mechanism.
